(19) United States
(12) Patent Application Publication
(10) Pub. No.: US 2016/0122703 A1
(43) Pub. Date: May 5, 2016

(54) METHOD AND SYSTEM FOR TREATING ORGANIC WASTE AND WASTEWATER

(71) Applicant: Robert Bernard Levine, Ann Arbor, MI (US)

(72) Inventor: Robert Bernard Levine, Ann Arbor, MI (US)

(21) Appl. No.: 14/874,041

(22) Filed: Oct. 2, 2015

Related U.S. Application Data
(60) Provisional application No. 62/074,030, filed on Nov. 2, 2014.

(51) Int. Cl. C12M 1/33; B02C 18/00; C02F 3/28; C02F 1/00; C12M 1/12; C12M 1/00

(52) U.S. Cl. CPC C12M 45/02; C12M 25/14; C12M 29/00; C02F 3/2806; C02F 3/2866; C02F 1/004; B02C 18/0092; C02F 2103/005

(57) ABSTRACT

An organic waste and wastewater treatment method and system that quickly and cost-effectively removes most organic materials from a waste or wastewater while generating a gaseous byproduct that can be used for heat or electricity generation. The method and system begins with a maceration and/or screening step that reduces waste particle size. Then, the waste is pumped through orifice(s) under high pressure to emulsify the waste and covert it to a slurry. The slurry is then treated in a horizontal anaerobic digester with flexible support material for microbial attachment to remove organic materials.

### CROSS-REFERENCE TO RELATED APPLICATIONS

This application claims the priority benefit of U.S. Provisional Application 62/074030 filed Nov, 2, 2014, which is herein incorporated by reference in its entirety.

### TECHNICAL FIELD

The present disclosure relates to organic waste disposal and treatment. More particularly, the disclosure discusses a system for treating organic wastes that reduces waste particle size prior to entering an anaerobic digester.

### BACKGROUND

The statements in this section merely provide background information related to the present disclosure. Accordingly, such statements are not intended to constitute an admission of prior art.

Effective organic waste management is becoming increasingly important in light of its significant environmental impacts as well as its rising cost to businesses. Each year, the US generates about 250 million tons of municipal solid waste, of which about 15% is energy and nutrient-rich food waste. These organic wastes are especially problematic as they degrade in landfills to produce methane, a greenhouse gas that is 23 times more powerful than carbon dioxide. According to the EPA, landfills are the third largest anthropogenic source of methane emissions in the US. In response to the negative environmental effects of landfilling, there is an emerging trend towards US municipalities and states completely banning the landfilling of food waste. In addition to being detrimental to the environment, waste hauling and disposal are expensive. As a result of declining landfill capacity and increased local opposition to new landfills, tipping fees have increased on average about 10% each year for the last decade and hauling costs have been driven up by higher fuel prices and an increase in interstate hauling.

Businesses that produce about 50-4000 kg/day of organic waste, such as supermarkets, institutional cafeterias, hotels, large restaurants, amusement parks, and sports stadiums, are burdened by these rising costs and emerging regulations. Some businesses have looked for relief by segregating a portion of their organic waste and having it hauled to centralized composting or regional anaerobic digestion facilities. Composting is an environmentally preferable solution compared to landfilling, since it reduces GHG emissions, diverts waste from landfills, and captures the nutrient value of the waste. However, centralized composting is often limited to just pre-consumer fruit and vegetable wastes, requires frequent waste pickups to combat odors and pests, releases volatile organic compounds, and fails to harness the energy contained within organic discards.

Centralized digestion of food waste, which can be done at some wastewater treatment plants already operating digesters, is a good alternative to landfilling and has the potential to capture both the energy and nutrient value of waste. However, centralized digestion provides no reduction in the frequency or magnitude of waste hauling, fails to improve on-site waste storage conditions, and delivers little benefit to the waste producer since the energy generated is owned by the facility operator. In addition, most commercial digesters today do not operate efficiently; often times only 40-60% of the organic matter in waste is actually converted into biogas and the resulting digestate is full of nitrogen and phosphorous. While the nutrients in digestate may have some value, in many situations, the cost of storing, transporting, and treating this wastewater outweighs the financial benefits from the nutrients. As a result, there is a need to more efficiently process organic wastes and digestate to cost effectively extract its energy and nutrient value.

In addition to centralized alternatives to landfilling organic waste, some waste generators have sought out onsite waste management devices to reduce the cost of waste disposal. Today, these onsite solutions include waste dehydrators, waste-to-water devices, and in-vessel composters. Waste dehydrators typically use large amounts of electricity to heat up the waste material and evaporate water. Since organic waste is usually 75-95% water, this results in a large reduction in waste volume and therefore less material to haul away. Waste-to-water devices also use a large amount of electricity to heat up waste, as well as potable water, to break down organic wastes through microbial processes and dilute it sufficiently to be discharged to the local sewer. Lastly, in-vessel composters use large amounts of electricity to heat up waste in an aerobic environment, thereby accelerating the composting process. In some cases, additional microbes are added to these vessels and the process is referred to as an aerobic digester. Each of these onsite devices is a net energy consumer and is typically expensive to buy or lease as well as costly to maintain. Furthermore, waste-to-water devices consume a large amount of potable water simply to dilute the waste prior to sending it the local treatment plant, many of which will now levy surcharges or fines on this type of discharge. Finally, in the case of waste dehydrators and in-vessel composters, the user must regularly manage the effluent of these devices as well as the associated odors. For the aforementioned reasons, none of these onsite waste management solutions for organic waste are ideal for small to mid-size waste generators (about 50-4000 kg/day).

In contrast to these onsite devices that demand electricity, there has been a recent attempt to process organic wastes onsite using anaerobic digestion to produce electricity (US Patent 2011/02000954 A1). Onsite anaerobic digestion allows facility owners to capture the energy value of the waste they generate through the production and then combustion of a methane-containing biogas. In general, anaerobic digestion is an ideal waste management strategy given its ability to reduce waste volume and weight, degrade recalcitrant natural compounds (e.g., cellulose, lignin), reduce waste-borne pathogens, and produce methane along with a nutrient-rich digestate. Digestion begins when organic polymers, such as starch, proteins, and lipids, are hydrolyzed into simple soluble compounds that can be absorbed by bacterial cells. After hydrolysis, acidogenesis occurs during which fermentative bacteria convert these monomers into low-molecular weight organic acids and alcohols, such as propionate and ethanol. During acetogenesis, these fermentation products are oxidized to acetate, carbon dioxide and hydrogen by acetogens. As a final step, methanogens convert these intermediates into methane gas. Through the combined activity of hydrolysis, acidogenesis, acetogenesis, and methanogenesis, a biogas is produced that is typically 50-60% methane, 40-50% carbon dioxide, and contains trace amounts of hydrogen sulfide.

The renewable energy microgeneration process described in US02000954 comprises a mixing tank with attached macerating pump, multiple small holding tanks for carrying out aerobic or anaerobic thermophilic digestion, one large holding tank for mesophilic anaerobic digestion, a dewatering unit, a controller that automates the flow of material between the tanks, and a portable gas storage container. While this system is designed to digest waste to produce a biogas, it is comprised of multiple tanks operated at different temperatures, several separate containers, multiple pumps and mixing devices, and a dewatering system that adds cost and complexity to the process. In addition, the system requires that waste be loaded into a hopper inside the container that is accessible only by opening the outer container door, making it inconvenient to use. Furthermore, the system requires that waste be diluted with water (typically 1:4 ratio of waste-to-water to achieve 8-10% total solids in the mixture), either using potable water or recovered liquid effluent from the dewatering device. This dilution of the waste feedstock leads to larger tank volumes required (about 4 times larger) and can result in a large consumption in potable water. Finally, the dewatering system continuously produces a separate solid and liquid fertilizer product that can produce unwanted odors and be difficult for waste generators (e.g., supermarket owners or cafeteria managers) to manage on a daily basis as well as transport offsite. For these aforementioned reasons, the previous attempt at onsite anaerobic digestion is not ideal for small to mid-size food waste generators (about 50-4000 kg/day) whose principle business is related to food service operations and not waste management. Clearly, there is a need for a simple and effective onsite solution that reduces the cost and environmental impact of organic waste disposal.

Just as food waste generating businesses require a new solution for onsite waste processing, farms too require assistance in sustainably managing animal wastes (e.g. manures) and other farm wastewaters (e.g., parlor wastewater). This is particularly important on dairy, swine, and chicken farms that are confined animal feeding operations and scrape or flush manure into pits and/or lagoons for storage and eventual spreading onto nearby fields. Manure spreading can be an expensive process, especially if it must be transported many miles from the farm for spreading, and results in the loading of significant amounts of nitrogen, phosphorus, and coliform bacteria onto the ground that can leach into waterways and cause eutrophication and dead zones, as well as poisoning of drinking water wells with nitrates and coliform bacteria. The storage of manure in lagoons also results in the release of methane, a potent greenhouse gas.

Attempts to treat manure onsite at farms with anaerobic digesters has been limited to very large farms that can afford expensive systems (typically >$5,000/animal all-in cost for a new digester). These digesters tend to be large above ground tanks or in-ground, concrete tanks that have a 20-60 day capacity for storing manure in the absence of oxygen and capturing any methane that is released. For a variety of reasons, most farm digesters only remove about 25-50% of the total organic content of manure and generate an effluent product called digestate that still must be stored and land applied, typically by hauling and spreading. This results in high rates of truck traffic on the road, which has an environmental impact (diesel emissions), along with social impacts (road quality impacts, higher rates of spills and accidents). There is a strong need for a new solution that can anaerobically digest manures onsite in a more cost-effective, quicker, and highly efficient manner.

A similar situation exists at municipal wastewater treatment facilities and other human sewage treatment facilities that produce sludge requiring disposal. The dewatering, drying, and transportation of sludge can be very expensive, not to mention energy intensive. While anaerobic digestion of sludge from sewage treatment has been practiced, it tends to require large, expensive systems with a residence time of at least 20-30 days and is suitable only at large facilities. There is a need for a lower-cost, more efficient, and scalable treatment technology for this type of organic waste.

One concept to reduce the cost of anaerobic digestion is to provide a media inside the digester upon which microbes can attach. By forming biofilms, the microbes responsible for consuming organic waste and producing methane remain within the digester instead of being removed with each's day effluent. While several attempts have been made to place media inside anaerobic digesters, many do not work well with organic wastes containing suspended solids (e.g., food waste, manures, etc.), clog rapidly (requiring expensive maintenance and/or replacement), and are excessively expensive. For example, stationary materials like gravel, other types of rock, corrugated pipes, and plastic packing with a high surface area to volume ratio (e.g. Pall rings) have all been employed in anaerobic treatment systems for various types of wastes. However, all these biofilm support materials eventually clog due to biofilm overgrowth and none can be effectively used with wastewaters high in suspended solids (such as food wastes and manures). Therefore, the present invention is a new method to treat waste in an anaerobic digester containing media that promotes microbial attachment (i.e. biofilm growth) but does so in a manner that is compatible with wastes containing solids and is cost-effective and uniquely clog-resistant.

### BRIEF SUMMARY OF THE INVENTION

The present technology includes systems, processes, articles of manufacture, and compositions that relate to disposing of and treating organic wastes. The present invention is a method for disposing of organic waste that produces a greywater effluent with very low levels of suspended solids, organic matter (measured as volatile solids or chemical oxygen demand or biological oxygen demand), and pathogens (measured as total coliforms and E. coli). The present invention is also useful to extract the energy content of the waste in the form of renewable natural gas or biogas with a high methane content (>60%). The present invention can dispose of waste cost effectively compared to alternatives available today, remain in service for long periods of time without maintenance, and achieves higher levels of treatment in a shorter amount of time. Use of this device will enable businesses that produce or handle organic wastes, animal manures, and other high-strength wastewaters to reduce waste-related greenhouse gas emissions, generate value from renewable energy use and sales, diminish their dependency on fossil fuels, and use water more efficiently.

In one embodiment, a method of treating organic wastes comprises: macerating the waste mechanically to eliminate solids greater than about 1 millimeter in diameter and/or screening the waste to remove solids greater than about 1 millimeter in diameter; pumping the waste through one or more orifice(s), wherein the absolute pressure inside the orifice(s) is less than atmospheric pressure, further wherein the orifice(s) is configured to emulsify the waste and convert it to a slurry; and pumping or allowing to flow by gravity the slurry through a horizontal anaerobic digester, wherein the digester comprises tubes or channels and each tube or channel has a cross-sectional width, further wherein at least one tube or channel within the digester contains at least one hanging device attached to the tube or channel and the hanging device holds a flexible support material upon which microbes can attach and grow.

In one embodiment, the slurry travels a total length through all of the tubes or channels and the total length is at least 10 times greater than a cross-sectional diameter or width of each tube or channel. The tubes or channels can be connected in series.

The waste can be of food scraps, human sewage, sludge from aerobic wastewater treatment plants, manure, digestate, fats, oils, greases, food processing wastewaters, or the like, or some combination thereof.

The macerating step can be performed with a disposer or the like.

The screening step can be performed with a screw-press separator, vibratory screen, or a passive screening/filter device, or the like.

The pumping the waste through one or more orifice(s) step is performed with a piston pump or the like.

The orifice(s) can be housed in an orifice container and the orifice(s) have a metal material of construction.

In one embodiment, the orifice(s) are 5 to 15 millimeters in diameter. The orifice(s) can have a circular, elliptical, rectangular, triangular, or the like cross-sectional shape.

In one embodiment, the slurry has an average hydraulic retention time of 0.5-15 days in the horizontal anaerobic digester.

In one embodiment, the slurry has an average hydraulic retention time of 3-5 days in the horizontal anaerobic digester.

In one embodiment, the microbes are able to consume organic material and generate combustible gas.

The support material can have a material of construction that is essentially plastic, textile, burlap, woven fabric, string, rope, hardened plant material, hemp, or the like, or some combination thereof.

In one embodiment, the hanging device(s) is placed perpendicular to a direction of slurry flow. In a separate embodiment, the hanging device(s) is placed parallel to a direction of slurry flow. In a separate embodiment, the hanging device(s) is placed at an angle 0-180 degrees to a direction of slurry flow.

In one embodiment, the waste is preheated prior to pumping the waste to the anaerobic digester. The waste can be preheated to a range of 60-150 degrees Fahrenheit.

In one embodiment, the anaerobic digester further comprises baffles or weirs configured to direct a slurry flow through the tubes or channels.

In one embodiment, the tubes or channels are made of concrete and/or plastic and are assembled together in series and have a total combined length at least 10 times greater than a width of each individual tube or channel.

In one embodiment, the slurry flows consistently or nearly consistently through the anaerobic digester 24 hours per day.

In one embodiment, the slurry flows in pulses through the anaerobic digester.

The slurry that flows through the digester can be roughly equal to an ambient temperature, above an ambient temperature, at 85-105 degrees Fahrenheit, at 85-150 degrees Fahrenheit, or at 130-140 degrees Fahrenheit.

In one embodiment, the digester comprises modules that can be assembled and connected on-site to create a continuous slurry flow. The digester can be configured for easy shipment in a standard shipping container or the like.

In one embodiment, the digester and/or the entire system can be partially buried in the ground.

In one embodiment, two or more digesters can be connected in parallel and a splitter used to divide the slurry between the digesters.

In one embodiment, more than about 80% of volatile solids in the waste are treated within an average hydraulic retention time of 5 days.

In one embodiment, more than about 80% of volatile solids in the waste are treated within an average hydraulic retention time of 3 days.

In one embodiment, more than about 80% of volatile solids in the waste are treated within an average hydraulic retention time of 1 day.

In one embodiment, more than about 80% of the total suspended solids in the waste are treated in less than 5 days average hydraulic retention time.

In one embodiment, more than about 80% of the total suspended solids in the waste are treated in less than 3 days average hydraulic retention time.

In one embodiment, more than about 80% of the total suspended solids in the waste are treated in less than 1 day average hydraulic retention time.

In one embodiment, the method further comprises generating a greywater effluent after the last step and removing suspended solids in the greywater effluent with ultrafiltration.

In a separate embodiment, a solution comprising Cobalt, Nickel, Zinc, or some combination thereof is added directly to the digester or to the waste slurry during the pumping or allowing to flow by gravity the slurry through a horizontal anaerobic digester step.

In one embodiment, a system for treating organic wastes comprises: a maceration and/or screening device configured to mechanically macerate waste solids to less than 1 mm particle size diameter and/or screen the waste solids to remove solids greater than 1 mm in diameter; a pump configured to pump the waste solids from the maceration and/or screening device; a housing containing one or more orifice(s) sized such that an absolute pressure inside the orifice is less than an atmospheric pressure when receiving the waste solids from the pump, wherein waste solids are emulsified and homogenized after passage through the orifice(s) and converted to a waste slurry; and a horizontal anaerobic digester which comprises tubes or channels and each tube or channel has a cross-sectional width, further wherein at least one tube or channel has at least one hanging device attached to the tube or channel and the hanging device holds a flexible support material upon which microbes can attach and grow.

In one embodiment, a system for treating organic wastes comprises: a pump configured to pump a macerated or screened waste at pressures between 100 and 1000 psi; and an orifice housing comprising one or more orifice(s) sized to emulsify and homogenize the waste and disintegrate particulate solids, wherein an absolute pressure inside the orifice(s) at a flow rate generated by the pump is less than an atmospheric pressure.

The scope of the invention is defined by the claims, which are incorporated into this section by reference. A more complete understanding of embodiments on the present disclosure will be afforded to those skilled in the art, as well as the realization of additional advantages thereof, by consideration of the following detailed description of one or more embodiments. Reference will be made to the appended sheets of drawings that will first be described briefly.

### BRIEF DESCRIPTION OF THE DRAWINGS

A clear understanding of the key features of the invention summarized above may be had by reference to the appended drawings, which illustrate the method and system of the invention, although it will be understood that such drawings depict preferred embodiments of the invention and, therefore, are not to be considered as limiting its scope with regard to other embodiments which the invention is capable of contemplating. Accordingly:

FIG. 1 shows a flow diagram of a system embodiment.

FIG. 2 shows a hanging device with hanging support material.

FIG. 3 shows modular digesters connected in series.

FIG. 4 shows a cut-away view of a digester embodiment.

FIG. 5 shows a perspective view of a cylindrical tube embodiment.

FIG. 6 shows a cut-away view of cylindrical tube embodiment.

FIG. 7 shows a cross-section view of flow through an orifice.

FIG. 8 shows a cut-away perspective view of a cylindrical tube digester channel.

FIG. 9 shows a cut-away perspective view of a rectangular digester channel.

FIG. 10 shows a cross-section side-view of a rectangular digester channel.

FIG. 11 shows a cross-section perspective view of a rectangular digester channel.

FIG. 12 shows a top perspective view of a digester channel embodiment.

FIG. 13 shows a top perspective view of two digester channels in one digester module.

FIG. 14 shows a perspective cut-away view of a single rounded channel embodiment.

FIG. 15 shows a perspective cut-away view of a single rectangular channel embodiment.

FIG. 16 shows a perspective cut-away view of a single rectangular channel embodiment with a hanging device oriented parallel to slurry flow.

FIG. 17 shows a flow diagram of a simplified system embodiment.

FIG. 18 shows a method embodiment.

### DETAILED DESCRIPTION

The following detailed description of the invention is merely exemplary in nature and is not intended to limit the invention or the application and uses of the invention. Furthermore, there is no intention to be bound by any theory presented in the preceding background of the invention or the following detailed description of the invention.

As discussed in the background, the present invention is a waste disposal and treatment system that removes the majority of the organic material in wastewaters and produces a gaseous product useful in the generation of heat and/or electricity. The inventor has discovered that organic waste can be cost-effectively and quickly treated through a combination of particle size reduction and high-rate (i.e. short residence time) anaerobic digestion when a flexible material is provided within the digester for microbes to attach to. The system can be readily scaled to any size and tuned to achieve different levels of performance. It can also be totally containerized for ease of transport and deployment.

The term “about” includes and describes the value or parameter per se. For example, “about x” includes and describes “x” per se. In certain embodiments, the term “about” when used in association with a measurement, or used to modify a value, a unit, a constant, or a range of values, refers to variations of +1-10%. In some embodiments, the term “about” when used in association with a measurement, or used to modify a value, a unit, a constant, or a range of values, refers to variations of +5%. In some embodiments, the term “about” when used in association with a measurement, or used to modify a value, a unit, a constant, or a range of values, refers to variations of +10%.

The term “and/or” includes subject matter in the alternative as well as subject matter in combination. For instance, “x and/or y” includes “x or y” and “x and y”.

It is understood that aspects and embodiments of the invention described herein including “consisting of and/or” “consisting essentially of” aspects and embodiments.

As used herein, the singular forms “a,” “an,” and “the” include the plural reference unless the context clearly dictates otherwise.

In one embodiment, the first step of the present invention is to reduce the particle size of the organic waste material to about less than 1 mm. This can be accomplished by macerating the organic waste to reduce the size of particles present in the waste to about less than 1 mm and/or removing particles larger than about 1 mm. In one embodiment, maceration can be accomplished with a garbage disposer consisting of one or more rotating elements that cut waste into smaller pieces. In another embodiment, a hammer mill or similar device can be used to macerate waste. A small amount of water is typically required to move waste through the macerator depending on the moisture content of the waste. In another embodiment, waste can be macerated with a grinder pump. In another embodiment, waste can be screened to remove particles larger than about 1 mm by using a screw-press separator or disc press or any similar equipment. Passive solids-separators (e.g., sloped screens) can be used as well. Some wastes may not require screening or macerating if all solids are already smaller than about 1 mm.

The remaining particles less than about 1 mm are then disintegrated and the waste slurry is homogenized and emulsified. Remaining solid particles are broken down into smaller particles and/or solubilized into the liquid phase and the waste becomes a more homogenous mixture. The waste slurry is pumped through a nozzle with one or more orifices such that the waste stream is accelerated to sufficiently high velocities to cause the absolute pressure in the orifice(s) to be reduced below atmospheric pressure (14.69 psi at sea level). Upon exiting the nozzle, the absolute pressure returns to some amount equal to or greater than atmospheric pressure. Without being bound by theory, the material is exposed to very high shearing forces as it passes through the orifice(s) under these conditions. The inventor discovered that unlike previous attempts to use orifice(s) for waste disintegration, which have focused on small orifices (<2 mm) and multiple passes of the waste through the orifice, a single pass through a larger orifice (>5 mm) achieves superior performance while using less energy and with a lower risk of clogging. Particle size reduction of waste solids in the orifice(s) helps accelerate microbial breakdown of the waste and emulsification and homogenization helps reduce settling in the anaerobic digester, which reduces maintenance and costs. In one embodiment, the pump used is a piston pump. In another embodiment, the waste is pumped at about 100 to 1000 psi (measured upstream of the orifice). Most preferably, the waste is pumped at 250 to 500 psi (measured upstream of the orifice). In another embodiment, the nozzle is crafted from a hard metal such as stainless steel.

The disintegrated waste slurry is then pumped or flows by gravity through an anaerobic digester containing support material upon which microbes can attach. In one embodiment, the digester is oriented horizontally such that the waste travels a total distance at least 10 times greater than the cross-sectional diameter (for a cylindrical shaped vessel) or width (for a rectangular-shaped vessel) of its flow path. The digester contains one or more channels in fluid contact with one another through which the waste travels.

The waste travels through the anaerobic digester for between 0.5 and 15 days average hydraulic retention time. In one embodiment, the waste travels through the anaerobic digester for between 3 and 5 days average hydraulic retention time. During this time, waste is hydrolyzed and then consumed by microbes.

In one embodiment, the digester is a constructed out of sections of round pipe, such as high density polyethylene (HDPE) or concrete pipe. In another embodiment, the anaerobic digester is constructed out of concrete poured in place or pre-cast in a rectangular shape to form channels. Overflow or underflow weirs and/or baffles placed in the tubular sections of pipe or rectangular sections of concrete channels reduce the chance of short-circuiting of waste materials. In one embodiment, sections of the digester are pre-formed and pre-assembled offsite, delivered onsite, and then quickly assembled by connecting the output of one module to the inlet of the next module. In another embodiment, the digester is made of concrete and poured in place at the site. The anaerobic digester can be above ground or below ground and optionally insulated. In one embodiment, the headspace of one or more channels or tubes is connected so there is gas mixing between each channel. In another embodiment, gas is collected separately from each channel or tube. The digester can be heated by hot fluid circulated within pipes located inside the walls of the digester or in fluid contact with the waste inside the digester and is optimally maintained around 100° F. In another embodiment, the digester is maintained at about 135° F.

The anaerobic digester is partially or completely filled with one or more support materials designed to retain anaerobic microbes within the reactor. Unlike previous attempts to immobilize biomass inside digesters, which have commonly failed due to microbial overgrowth and solids plugging that requires expensive maintenance, this invention utilizes a flexible support material hung vertically from hanging devices such as a beam. Without being bound by theory, the inventor discovered that movement of the support material, caused by flow of the waste through the digester and/or by rising gas bubbles, helps to reduce overgrowth of the microbial biofilm and diminishes the chance of clogging. In one embodiment, the material is flat, rectangular-shaped strips of plastic, such as high density polyethylene, that hang from a support beam mounted across the top of the digester channel. The strips can be about 0.25 to 24 inches wide and any length but preferably extend at least the entire liquid height of the digester channel to maximize the surface area of the material in contact with the waste. The strips can optionally be hung from the hanging device so that each strip does not overlap an adjacent strip, each strip somewhat overlaps an adjacent strip, or strips substantially overlap one another. The strips are preferably a flexible material but can also be inflexible so long as the attachment mechanism to the hanging device is hinged and allows the material to move (i.e., swing back and forth). The back and forth motion of the flexible support material in any configuration can be modulated by the flow rate of material through the digester. For example, waste can be pumped into the digester for 20 min every 30 min, causing a pulsation effect that moves the flexible support material. The strips can be enhanced with a bead of plastic or similar material and/or folded and/or creased in any way that further increases its surface area. In another embodiment, the hanging device can be mounted in the headspace of the digester so that the same support material can suspend material both in the headspace and in the liquid, thereby providing material upon which hydrogen-sulfide reducing bacteria can grow in the headspace to help remove hydrogen sulfide gas from the gas output of the digester. In another embodiment, the material is a woven or perforated material made from plastic, webbing, cloth, hemp, burlap, or a similar material. In another embodiment, the material is cylindrical shaped such as a thick string or rope or similar woven material. The material can be affixed to the hanging device as an individual piece that is the appropriate length for the height of the digester channel and/or the material can be twice the length of the height of the digester channel and laid over the top of the hanging device so an equal length of the material hangs on both sides of the hanging device. The inventor has discovered that the free movement of the material within the digester channel is critical to its long-term, clog-free operation.

The hanging device that holds the material upon which microbes attach can be made of any suitable material known to those skilled in the art. In one embodiment, wood and/or plastic and/or metal can be used. In another embodiment, the support beam is constructed of cable or rope. The support beams can be affixed to the side walls of the digester using hangers or other devices known to those skilled in the art. In one embodiment, the support beam is easily removable so that it can be taken out of the digester for servicing if required. This makes it easy to lift up a section of support material quickly.

The hanging devices can be spaced at any interval within the digester channel to change the amount of surface area of material for biomass attachment exposed to the waste. In one embodiment, hanging devices with hanging support material are spaced perpendicular to the direction of waste flow about 1″ to 10′ apart in a digester channel. In another embodiment, the hanging devices are placed parallel to the direction of flow and are spaced about 1″ to 10′ apart in a digester channel. In one embodiment, the spacing between the hanging devices is consistent throughout the digester. In another embodiment, the spacing between the hanging devices changes throughout the digester. For example, the spacing between hanging devices can diminish along the length of the digester such that the surface area of material available for microbial attachment increases as the waste becomes more digested. Conversely, the spacing between hanging devices can increase along the length of the digester such that the surface area of material available for microbial attachment decreases as the waste becomes more digested.

The high density of microbial biomass retained by the support material helps treat the waste quickly because microbes are not being washed out with the effluent of the digester. Therefore, overall, the waste is exposed to a higher concentration of microbes than in a typical plug-flow or completely mixed digester and as a result treatment is accomplished more rapidly. Any accumulated solids, typically highly mineralized, can be collected on the bottom of the digester channel and pumped out. Due to the movement of the flexible material within the digester, these mineralized solids do not clog the media but rather settle to the bottom of the digester channel. In one embodiment, the top of the digester channels contain access ports for cleaning. In another embodiment, the access ports for cleaning are attached to pipes that reach to the bottom of the digester channel (under the liquid level) so it can be vacuumed and/or pumped out without needing to evacuate the whole digester of gas. In another embodiment, the first channel within a digester with multiple channels contains overflow weirs but no hanging devices with support material so any accumulated solids can be readily cleaned out prior to the waste entering channels containing hanging devices.

Organic carbon is removed from the waste slurry through microbial action, creating a greywater effluent with very low biological oxygen demand (BOD), chemical oxygen demand (COD), and volatile solids (VS). A gaseous product is generated that contains a mixture of carbon dioxide and methane. In one embodiment, the content of methane is optimized so that it is higher than 60% and preferably higher than 70% of the total gas volume and most preferably higher than 80% of the total gas volume. This gaseous product can be combusted in any equipment suitable for the task, such as a generator, combined heat and power system, water heater, boiler, etc.

The gaseous product also contains small amounts of hydrogen sulfide gas. It is desirable to remove hydrogen sulfide from the biogas to reduce wear and corrosion of combustion devices. By hanging the support material partially in the headspace of the digester, hydrogen sulfide reducing bacteria can attach to the material and help reduce the amount of hydrogen sulfide in the biogas that exits the digester. By using a single support material that extends from the headspace to the liquid phase, water and nutrients can diffuse along the length of the material to supply the bacteria in the headspace with necessary nutrients. In one embodiment, hydrogen sulfide removal with media suspended in the headspace is accelerated by the addition of air to the digester in an amount about 1-10% of the total biogas flow rate (i.e. if the digester produced 100 SCFM of biogas one may add between 1 and 10 SCFM of air).

The waste treatment system described herein is substantially different from other waste treatment systems in that: 1) Waste particle size is first reduced and then waste is homogenized and emulsified prior to treatment by anaerobic microbes. 2) The flow of waste is orientated horizontally within one or more channels within an anaerobic digester and the waste must pass through a vertically-oriented flexible hanging material upon which microbes attach. 3) The digester contains a mechanism by which to remove settled solids/sludge when desired through ports in the digester without disturbing the gas headspace, making clean out easy.

### EXAMPLE 1

Micronizer system for dairy manure. Dairy manure was prepared for digestion by first pumping it through a screw-press containing a screen with 750-micron perforations to remove large solid particles. The liquid effluent of the screw-press, called pressate, was then pumped with a piston pump through a nozzle containing a single orifice sized such that the absolute pressure in the orifice was less than atmospheric pressure. In order to minimize nozzle fouling and energy consumed for pumping, the flowrate was about 40 GPM and the orifice size was about 7.9 mm. The material exited the nozzle and was collected in a 250 gallon tote at atmospheric pressure. The pressate and effluent from the nozzle was tested to determine its dry matter (DM), organic dry matter (oDM), total suspended solids (measured with a 20-25 micron filter paper or 1.5 micron filter paper), biological oxygen demand (BOD, measured in the whole sample), and chemical oxygen demand (COD, measured in the whole sample or in the filtrate after filtering the sample with a 20-25 micron filter paper or a 1.5 micron filter paper).

As shown in Table 1 below, the nozzle had very little if any effect on the dry matter and organic dry matter content of the manure pressate, as was expected. The nozzle, however, had a dramatic effect on the size of the suspended solids and partitioning of the organic material. The total amount of suspended solids measured with a 20-25 micron or 1.5 micron filter paper decreased about 7.5% or 14.2%, respectively, after passing through the nozzle. This means particles that were bigger than 20-25 microns or 1.5 microns were no longer retained on the filter paper, suggesting that they were reduced in size or solubilized into the liquid phase. There was about a 9-10% reduction in the total biological and chemical oxygen demand after the pressate passed through the nozzle, likely indicating that some oxidation of organic material occurred as a result of the forces generated during passage through the orifice. When the pressate was filtered with a 20-25 micron filter paper, the filtrate (liquid that passes through the filter paper) contained 17,689 mg/L COD whereas after passing through the nozzle the filtrate contained 31,863 mg/L COD. These data reveal that the nozzle significantly increased the concentration of organic material in the fraction smaller than 20-25 microns. A similar trend was observed by filtering with a 1.5 micron filter paper; filtrate from the sample that had passed through the nozzle contained 24,218 mg/L COD compared to only 7,543 mg/L COD in the raw pressate.

TABLE 1

| | | | Percent |
|---|---|---|---|
| | | | Change |
| | | | (Pressate to |
| | Raw | Nozzle | Nozzle |
| Parameter | Pressate | Effluent | Effluent) |
| Dry Matter (DM, %) | 5.10 | 5.0 | 1.96% |
| Organic Dry Matter (oDM, % of DM) | 73.4 | 74.7 | 1.77% |
| Total Suspended Solids (20-25 um filter, mg/L) | 32,258 | 29,821 | 7.55% |
| Total Suspended Solids (1.5 um filter, mg/L) | 35,864 | 30,769 | 14.21% |
| Biochemical Oxygen Demand (5-day, mg/L) | 12,463 | 11,225 | 9.93% |
| Chemical Oxygen Demand (Unfiltered, mg/L) | 50,810 | 46,210 | 9.05% |
| Chemical Oxygen Demand (Filtered 20-25 um, mg/L) | 17,689 | 31,863 | 80.13% |
| Chemical Oxygen Demand (Filtered 1.5 um, mg/L) | 7,543 | 24,218 | 221.07% |

### EXAMPLE 2

Anaerobic digester for 20,000 GPD of dairy manure. A digester was constructed to process 20,000 gallons per day (GPD) of dairy manure. The digester is 54′ long and 37′ wide and 10′-10″ high and is an in-ground concrete tank with 4 channels (each about 9′ wide) such that micronized waste travels through it in a series configuration. The total liquid volume of the digester is about 100,000 gallons, which corresponds to about a 5-day average retention time. The first channel contains three overflow weirs with no hanging devices and the second, third, and fourth channels contain hanging devices with hanging flexible plastic support material orientated perpendicular to the flow of waste spaced about 1′ apart. The microbial support material is comprised of plastic strips that are 1.25″ wide ×18′ long and hang over the support beam so that about 9′ of material hangs on either side of the beam. In total, the digester contains about 87,000 square feet of surface area of this material that microbes can attach to. This is about 10.8 ft²/ft³ of support material. The support material extends from the liquid phase into the gas headspace to help reduce hydrogen sulfide in the biogas. Biogas is collected from the digester and combusted in a boiler to provide heat to the digester via heating pipes integrated into the concrete walls as well as to other processes on the farm that require heat. The greywater effluent of the digester is processed by ultrafiltration to yield a liquid essentially free from suspended solids.

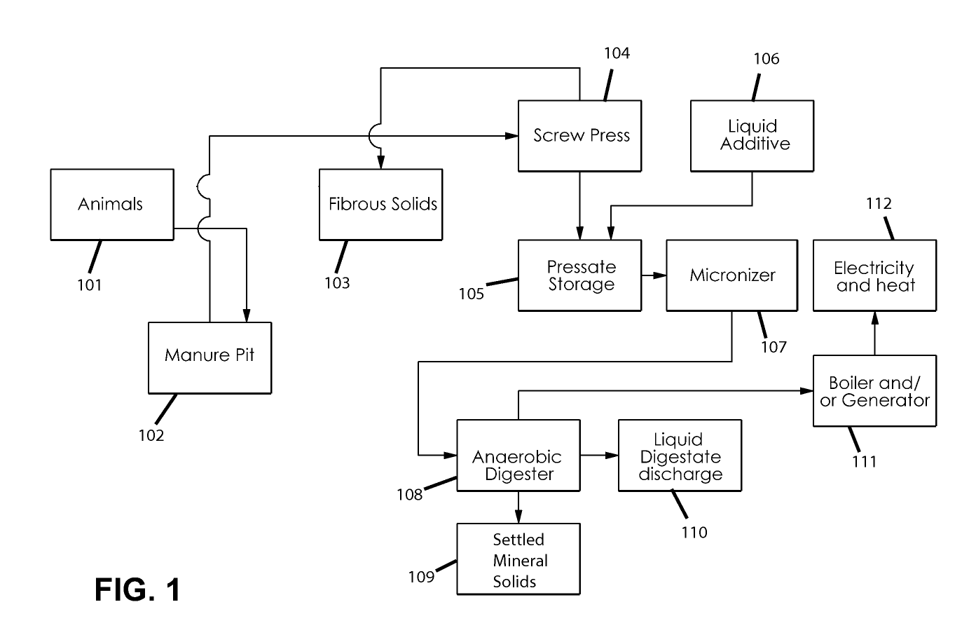

FIG. 1 shows a flow diagram of a system embodiment. Shown is waste generated by animals 101 that is collected in a pit 102 and then pumped to a screw press 104 which could also be replaced by a macerator or other device configured to reduce waste particulate size to less than about 1 mm in diameter. The screw press 104 removes solids larger than about 1 mm as fibrous solids 103. The waste with less than 1 mm particulate size then goes to pressate storage 105 where a liquid additive 106 such as water and/or minerals can be added to provide adequate levels of certain compounds required by anaerobic microbes to function optimally. From there, the slurry goes to a micronizer 107 such as a nozzle containing one or more orifices. The waste is pumped into the micronizer 107 at sufficiently high velocity such that the absolute pressure in the orifice(s) is less than atmospheric pressure. The waste solids are further disintegrated, emulsified, and solubilized in the micronizer. The forces within the micronizer can also catalyze chemical reactions within the slurry, further processing the waste slurry. From the micronizer 107, the slurry goes to an anaerobic digester 108. The slurry residence time can vary in the anaerobic digester 108 from 0-15 days, depending upon the strength of the waste and desired treatment. From there, liquid waste is generated as liquid digestate discharge 110, gases are sent to a boiler and/or generator 111 to convert the gases to electricity and heat 112, and some settled mineral solids 109 can be removed periodically as needed.

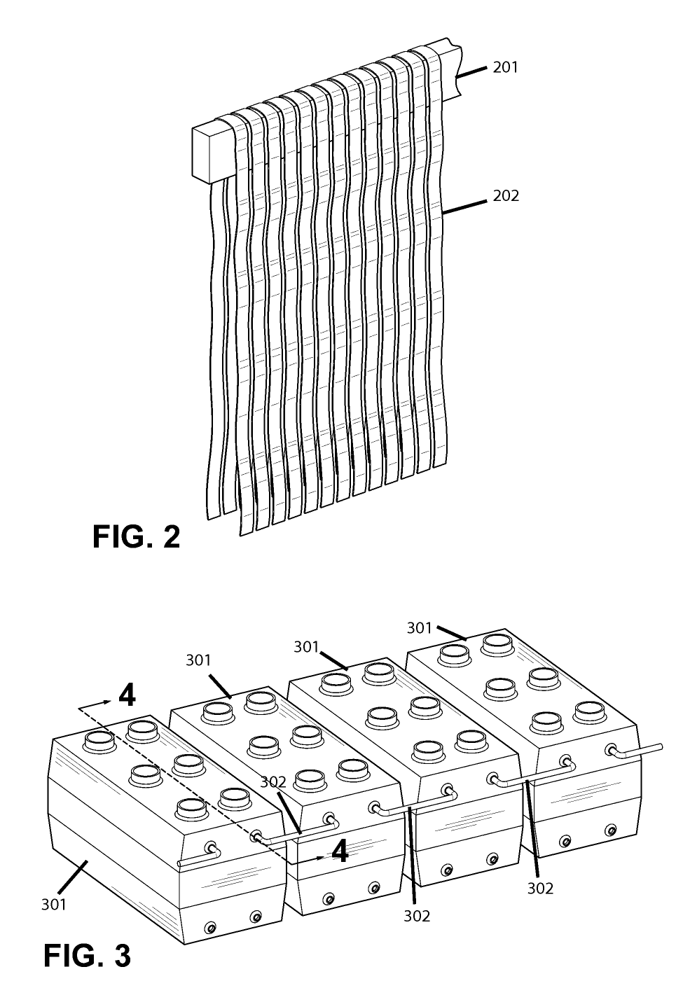

FIG. 2 shows a hanging device with hanging support material. Shown are a hanging device 201 such as a rod or the like and hanging support material 202 configured to retain microbes that are capable of digesting organic wastes. The hanging support material shown is flexible plastic that is about twice the length of the channel height and drapes over the hanging device.

FIG. 3 shows modular digesters connected in series. Shown are digesters 301 and piping connections 302.

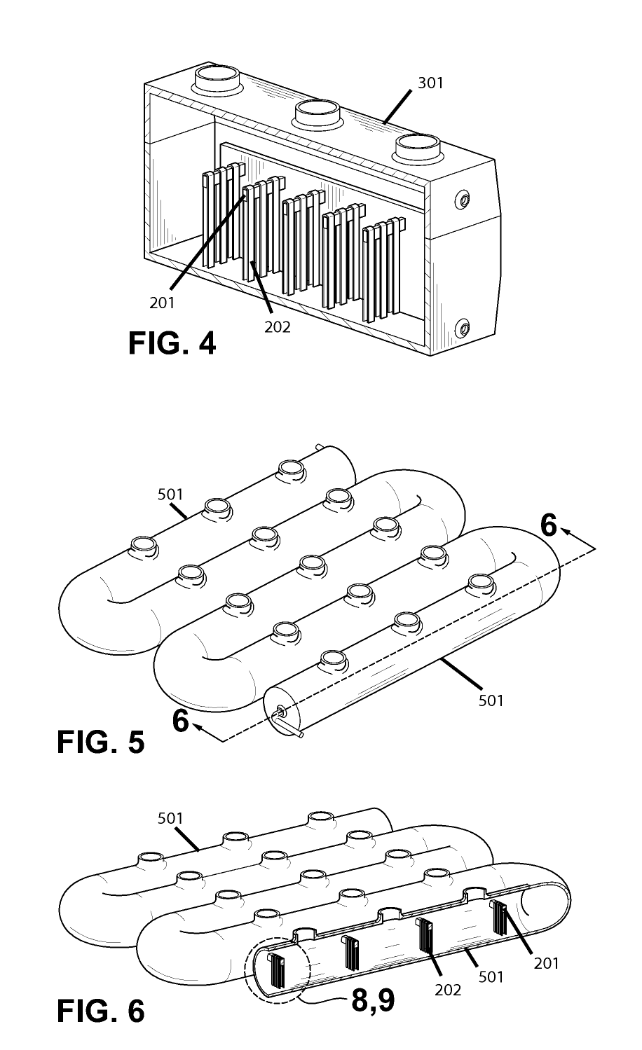

FIG. 4 shows a cut-away view of a digester embodiment. Shown are a digester 301, hanging device 201 and hanging support material 202.

FIG. 5 shows a perspective view of cylindrical tube embodiment. Shown are cylindrical tubes 501 connected in series.

FIG. 6 shows a cut-away view of cylindrical tube embodiment. Shown are cylindrical tubes 501 connected in series, hanging device 201, and hanging support material 202.

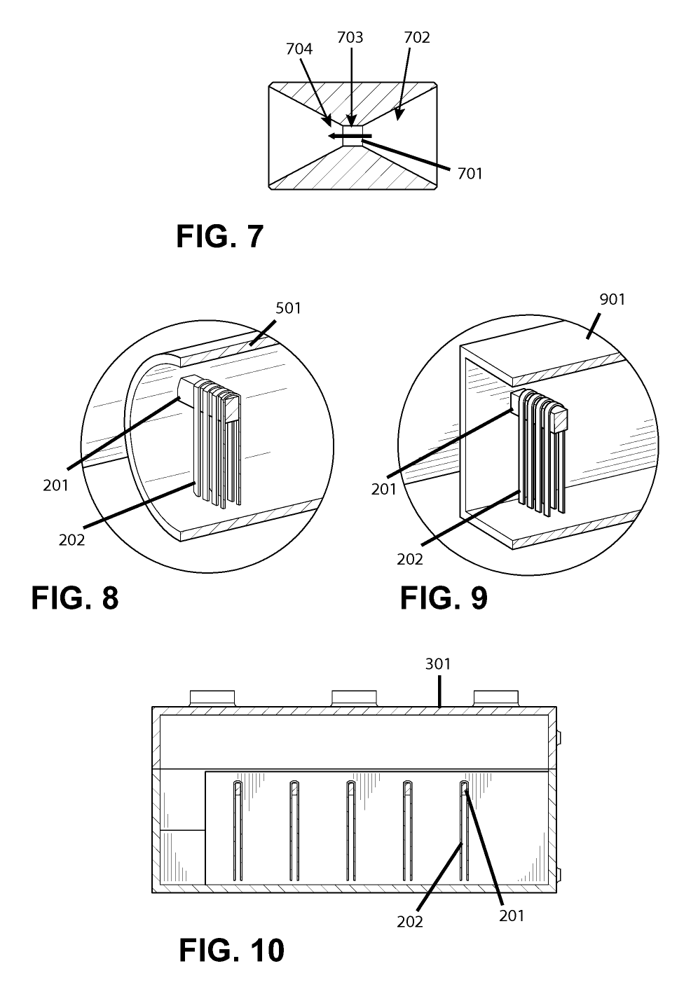

FIG. 7 shows a cross-section view of flow through an orifice. Shown is the orifice 701 with flow going through. A high pressure area 702 exists prior to the orifice and a low pressure area 703 exists within the orifice and secondary low pressure area 704 exists after the orifice.

FIG. 8 shows a cut-away perspective view of a cylindrical tube digester channel. Shown are cylindrical tube 501, hanging device 201, and hanging support material 202.

FIG. 9 shows a cut-away perspective view of a rectangular digester channel. Shown are rectangular channel 901, hanging device 201, and hanging support material 202.

FIG. 10 shows a cross-section side-view of a rectangular digester channel. Shown are digester 301, hanging device 201, and hanging support material 202.

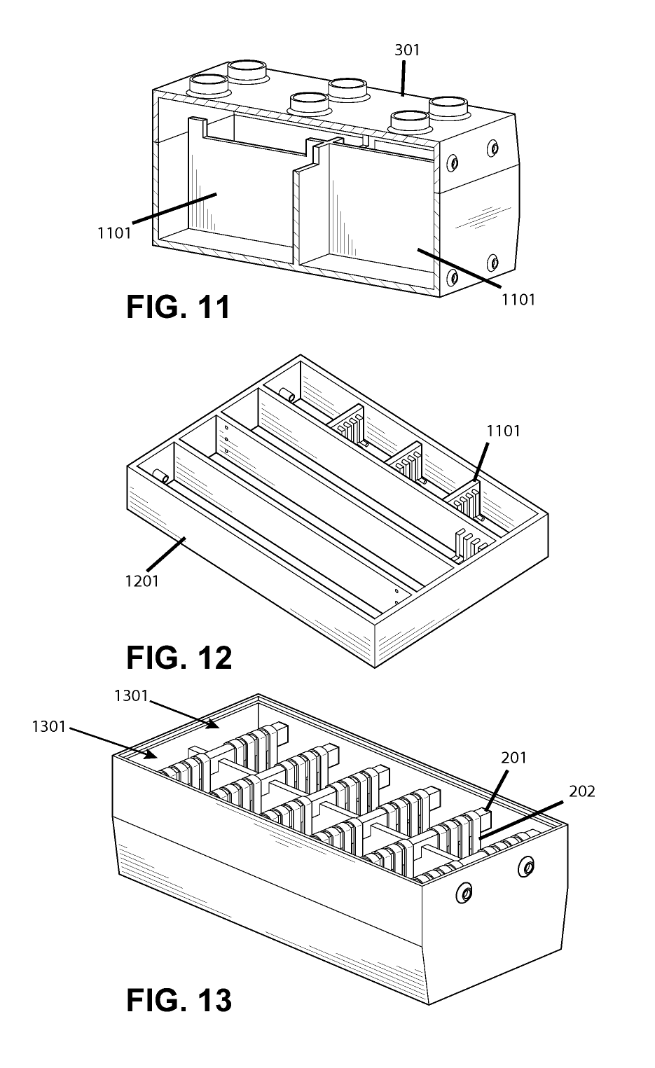

FIG. 11 shows a cross-section perspective view of a rectangular digester channel. Shown are digester 301 and weirs 1101.

FIG. 12 shows a top perspective view of a digester channel embodiment. Shown are a digester 1201 with four channels and three overflow weirs 1101 in the first channel.

FIG. 13 shows a top perspective view of two digester channels. Shown are digester channels 1301, hanging devices 201, and hanging support material 202.

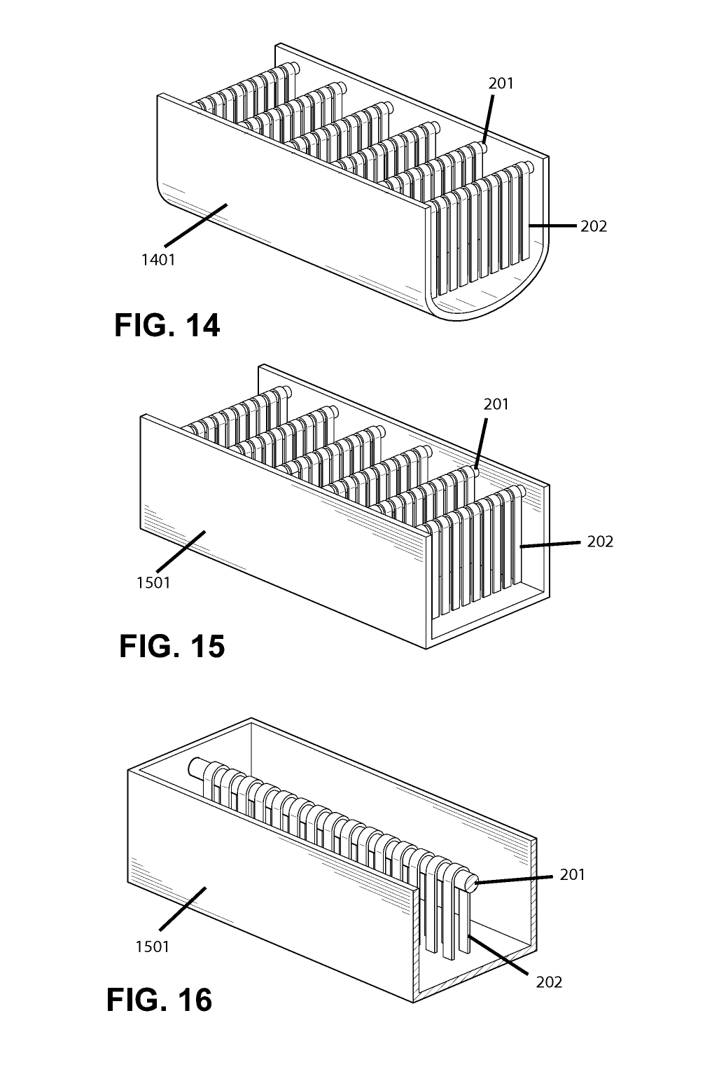

FIG. 14 shows a perspective cut-away view of a single rounded channel embodiment. Shown are rounded channel 1401, hanging devices 201, and hanging support material 202.

FIG. 15 shows a perspective cut-away view of a single rectangular channel embodiment. Shown are rectangular channel 1501, hanging devices 201, and hanging support material 202.

FIG. 16 shows a perspective cut-away view of a single rectangular channel embodiment with a hanging device oriented parallel to slurry flow. Shown are rectangular channel 1501, hanging device 201, and hanging support material 202.

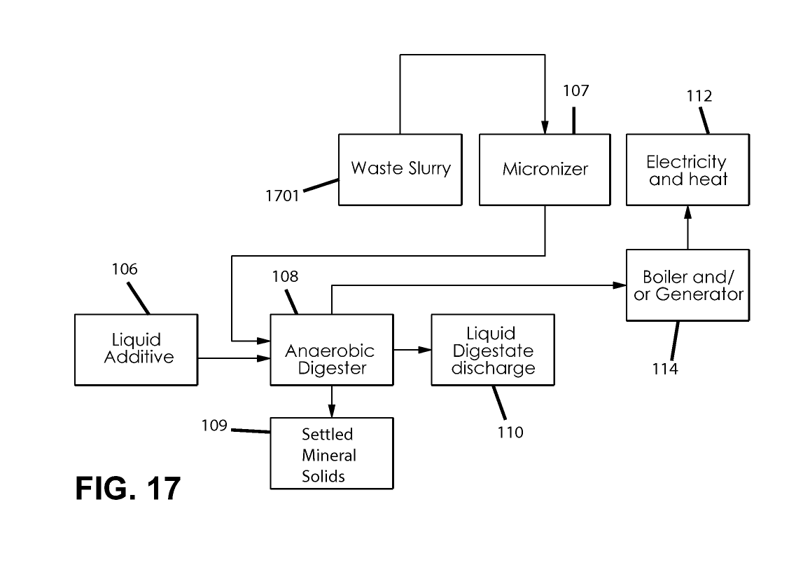

FIG. 17 shows a flow diagram of a simplified system embodiment. Shown are waste slurry 1701 (such as waste generated by animals 101) going into a micronizer 107 such as a nozzle containing one or more orifices. The waste is pumped into the micronizer 107 at sufficiently high velocity such that the absolute pressure in the orifice(s) is less than atmospheric pressure. The waste solids are further disintegrated, emulsified, and solubilized in the micronizer. The forces within the micronizer can also catalyze chemical reactions within the slurry, further processing the waste slurry. From the micronizer 107, the slurry goes to an anaerobic digester 108. The slurry residence time can vary in the anaerobic digester 108 from 0-15 days, depending upon the strength of the waste and desired treatment. From there, liquid waste is generated as liquid digestate discharge 110, gases are sent to a boiler and/or generator 111 to convert the gases to electricity and heat 112, and some settled mineral solids 109 can be removed periodically as needed.

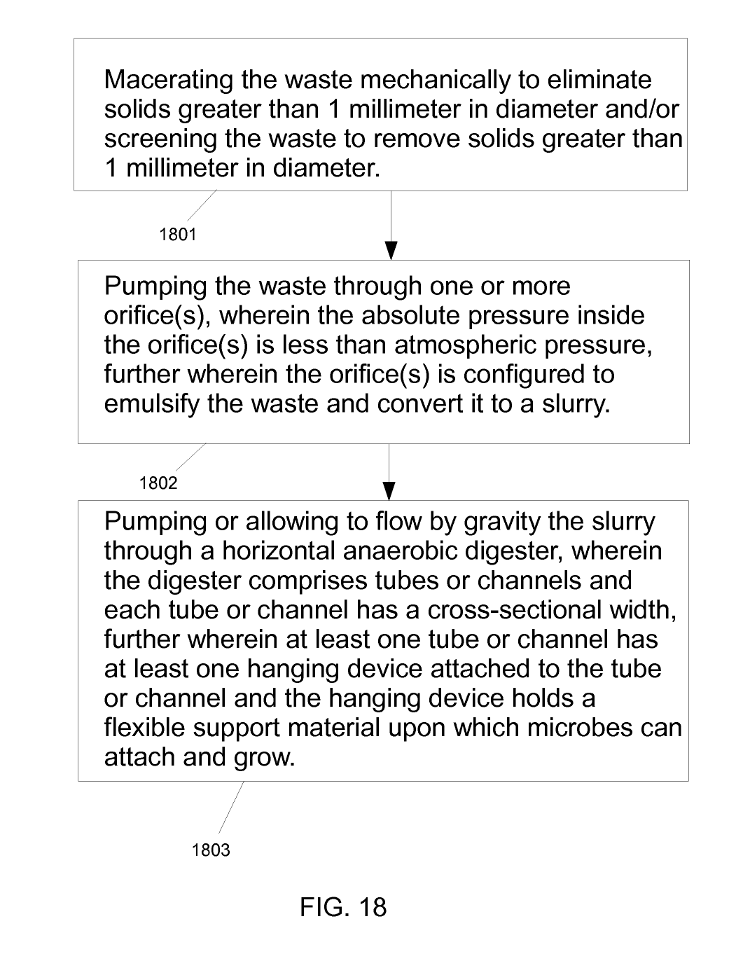

FIG. 18 shows a method embodiment. Shown are step 1801, macerating the waste mechanically to eliminate solids greater than 1 millimeter in diameter and/or screening the waste to remove solids greater than 1 millimeter in diameter; step 1802, pumping the waste through one or more orifice(s), wherein the absolute pressure inside the orifice(s) is less than atmospheric pressure, further wherein the orifice(s) is configured to emulsify the waste and convert it to a slurry; and step 1803, pumping or allowing to flow by gravity the slurry through a horizontal anaerobic digester, wherein the digester comprises tubes or channels and each tube or channel has a cross-sectional width, further wherein at least one tube or channel has at least one hanging device attached to the tube or channel and the hanging device holds a flexible support material upon which microbes can attach and grow.

All patents and publications mentioned in the prior art are indicative of the levels of those skilled in the art to which the invention pertains. All patents and publications are herein incorporated by reference to the same extent as if each individual publication was specifically and individually indicated to be incorporated by reference, to the extent that they do not conflict with this disclosure.

While the present invention has been described with reference to exemplary embodiments, it will be readily apparent to those skilled in the art that the invention is not limited to the disclosed or illustrated embodiments but, on the contrary, is intended to cover numerous other modifications, substitutions, variations, and broad equivalent arrangements.

I claim:

1. A method of treating an organic waste, the method comprising:
macerating the waste mechanically to eliminate solids greater than 1 millimeter in diameter and/or screening the waste to remove solids greater than 1 millimeter in diameter;
pumping the waste through one or more orifice(s), wherein the absolute pressure inside the orifice(s) is less than atmospheric pressure, further wherein the orifice(s) is configured to emulsify the waste and convert it to a slurry; and
pumping or allowing to flow by gravity the slurry through a horizontal anaerobic digester, wherein the digester comprises tubes or channels and each tube or channel has a cross-sectional width, further wherein at least one tube or channel has at least one hanging device attached to the tube or channel and the hanging device holds a flexible support material upon which microbes can attach and grow.

2. The method of claim 1, wherein the slurry travels a total length through all of the tubes or channels and the total length is at least 10 times greater than a cross-sectional diameter or width of each tube or channel.

3. The method of claim 1, wherein the waste is a plant or animal derived waste product selected from the group consisting essentially of food scraps, human sewage, sludge from aerobic wastewater treatment plants, manure, digestate, fats, oils, greases, and food processing wastewaters.

4. The method of claim 1, wherein the macerating step is performed with a disposer.

5. The method of claim 1, wherein the screening step is performed with a screw-press separator, vibratory screen, or a passive screening/filter device.

6. The method of claim 1, wherein the pumping the waste through one or more orifice(s) step is performed with a piston pump.

7. The method of claim 1, wherein the orifice(s) are housed in an orifice container and the orifice(s) have a metal material of construction.

8. The method of claim 1, wherein the orifice(s) are 5 to 15 millimeters in diameter.

9. The method of claim 1, wherein the slurry has an average hydraulic retention time of 0.5-15 days in the horizontal anaerobic digester.

10. The method of claim 1, wherein the slurry has an average hydraulic retention time of 3-5 days in the horizontal anaerobic digester.

11. The method of claim 1, wherein the microbes are able to consume organic material and generate combustible gas.

12. The method of claim 1, wherein the support material has a material of construction selected from the group consisting essentially of plastic, textile, burlap, woven fabric, string, rope, webbing, hardened plant material, hemp, or some combination thereof.

13. The method of claim 1, wherein the hanging device(s) is placed perpendicular to a direction of slurry flow.

14. The method of claim 1, wherein the hanging device(s) is placed parallel to a direction of slurry flow.

15. The method of claim 1, further comprising preheating the waste prior to the pumping the waste through one or more orifice(s) step.

16. The method of claim 1, further comprising preheating the waste prior to the pumping or allowing to flow by gravity the slurry through a horizontal anaerobic digester step.

17. The method of claim 1, wherein the anaerobic digester further comprises baffles or weirs configured to direct a slurry flow through the tubes or channels.

18. The method of claim 1, wherein the tubes or channels are made of concrete and/or plastic and assembled together in series and have a total combined length at least 10 times greater than a width of each individual tube or channel.

19. The method of claim 1, wherein the slurry flows consistently or nearly consistently through the anaerobic digester 24 hours per day.

20. The method of claim 1, wherein the slurry flows in pulses through the anaerobic digester.

21. A method of treating an organic waste, the method comprising:
pumping or allowing to flow by gravity a slurry through a horizontal anaerobic digester, wherein the digester comprises tubes or channels and each tube or channel has a cross-sectional width, further wherein at least one tube or channel has at least one hanging device attached to the tube or channel and the hanging device holds a flexible support material upon which microbes can attach and grow.

22. The method of claim 21, further comprising, prior to the pumping or allowing to flow by gravity step, pumping the waste through one or more orifice(s), wherein the absolute pressure inside the orifice(s) is less than atmospheric pressure, further wherein the orifice(s) is configured to emulsify the waste and convert it to a slurry.

23. A system for treating organic waste and wastewaters, comprising:
a maceration and/or screening device configured to mechanically macerate waste solids to less than 1 mm particle size diameter and/or screen the waste solids to remove solids greater than 1 mm in diameter;
a pump configured to pump the waste solids from the maceration and/or screening device;
a housing containing one or more orifice(s) sized such that an absolute pressure inside the orifice is less than an atmospheric pressure when receiving the waste solids from the pump, wherein waste solids are emulsified and homogenized after passage through the orifice(s) and converted to a waste slurry; and
a horizontal anaerobic digester which comprises tubes or channels and each tube or channel has a cross-sectional width, further wherein at least one tube or channel has at least one hanging device attached to the tube or channel and the hanging device holds a flexible support material upon which microbes can attach and grow.

24. The system of claim 23, wherein the slurry travels a total length through all of the tubes or channels and the total length is at least 10 times greater than a cross-sectional diameter or width of each tube or channel.

25. A system for emulsifying, homogenizing, and disintegrating organic waste and wastewaters prior to treatment, comprising:
a pump configured to pump a macerated or screened waste at pressures between 100 and 1000 psi; and
an orifice housing comprising one or more orifice(s) sized to emulsify and homogenize the waste and disintegrate particulate solids, wherein an absolute pressure inside the orifice(s) at a flow rate generated by the pump is less than an atmospheric pressure.
